(19) United States
(12) Patent Application Publication
(10) Pub. No.: US 2020/0207752 A1
(43) Pub. Date: Jul. 2, 2020

(54) ORGANIC ELECTROLUMINESCENT COMPOUND AND ORGANIC ELECTROLUMINESCENT DEVICE CONTAINING THE SAME

(71) Applicant: INT TECH CO., LTD., HSINCHU COUNTY (TW)

(72) Inventors: LI-CHEN WEI, TAICHUNG CITY (TW); MIN-HSIEN CHEN, PINGTUNG COUNTY (TW)

(21) Appl. No.: 16/232,638

(22) Filed: Dec. 26, 2018

(51) Int. Cl. C07D 417/14; H01L 51/50; H01L 51/00

(52) U.S. Cl. CPC C07D 417/14; H01L 51/5016; H01L 51/5096; H01L 51/0061; H01L 51/0072; H01L 51/5056; H01L 51/5072; H01L 51/5092

(57) ABSTRACT

The present disclosure provides an organic electroluminescent compound represented by the formula (1):

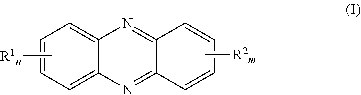

wherein each of R¹ and R² is independently an electron-donating group and each of n and m is independently an integer between 1 and 4. The present disclosure further provides an organic light-emitting device and an organic electroluminescent device each comprising the organic electroluminescent compound represented by the formula (I).

### FIELD OF INVENTION

The present invention relates to an organic electroluminescent compound and an organic electroluminescent device using the compound. More specifically, the present invention relates to an organic electroluminescent compound represented by Formula (I), and an organic electroluminescent device using the same.

### BACKGROUND

Organic light-emitting diode (OLED) devices have recently become very important to the scientific community and the display industry, and are now useful in displays such as television screens, computer monitors, mobile phones, and tablets.

However, the OLED device technology is currently experiencing an obstacle in the development process. A main issue is that light-emitting efficiency cannot meet practical demand, so the development of the OLED technology is greatly limited. One of the factors affecting the light-emitting efficiency of the OLED device is the efficiency of transporting carriers, including electrons and holes.

An OLED device includes a plurality of layers placed between a pair of electrodes and emits light in response to excitation such as by an electric current. The OLED device works on the principal that electrons and holes diffuse through an electron transport layer (ETL) and hole transport layer (HTL), respectively, to enter a light-emitting layer (EML), and recombine in the emitting region to form a particle generally referred to as an exciton. When an external voltage is applied to an OLED device, electrons and holes are injected from a cathode and an anode, respectively, Electrons are injected from a cathode into a lowest unoccupied molecular orbital (LUMO) and holes are injected from an anode into a highest occupied molecular orbital (HOMO). When the electrons recombine with holes in the EML, excitons are formed and the excitons then emit light. When luminescent molecules absorb energy to achieve an excited state, an exciton may be in either a singlet excited state (S₁) or a triplet excited state (T₁), depending on how the spins of the electron and the hole have been combined. Seventy-five percent of the excitons form by recombination of electrons and holes to achieve a triplet excited state. Decay from triplet states is spin forbidden, thus, a fluorescence electroluminescent device has only 25% internal quantum efficiency (IQE). In contrast to the fluorescent OLED device, a phosphorescent OLED device makes use of spin-orbit interactions to facilitate intersystem crossing between singlet and triplet states, thus obtaining emission from both singlet and triplet states and raising the internal quantum efficiency of OLED devices from 25% to 100%.

Thermally activated delayed fluorescence (TADF) materials are third-generation organic light-emitting materials, and were developed after the fluorescent materials and the phosphorescent materials. The energy gap between the singlet excited state and the triplet excited state (ΔE_{ST}) of the TADF materials is small, which allows excitons to transition from the triplet excited state to the singlet excited state through reverse intersystem crossing (RISC). Therefore, the TADF materials can take advantage of the excitons in singlet excited state and triplet excited state releasing energy in radiative form (fluorescence and delayed fluorescence), which enables the IQE of the TADF materials to reach to 100%. Accordingly, the TADF materials have received significant attention.

However, the OLEDs made of the TADF materials do not readily provide an external quantum efficiency (EQE) comparable to those of the phosphorescent materials. Research shows that the molecular structure of the TADF material is critical to the performance of the OLEDs. For example, it has been found that luminescent molecules with a small overlap between their HOMO bearing electron-donating groups and LUMO bearing electron-accepting groups can increase the TADF property.

Therefore, there is a demand for designing and developing novel TADF materials, in which the electron-donating groups and the electron-accepting groups are properly arranged, so as to provide the OLED device with a practical light emission efficiency and long service life.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a schematic cross-sectional view illustrating an electroluminescent element of an OLED in accordance with some embodiments of the present disclosure.

FIG. 2 is a schematic cross-sectional view illustrating an organic electroluminescent device in accordance with some embodiments of the present disclosure.

FIG. 3 is a schematic cross-sectional view illustrating an organic electroluminescent device in accordance with some embodiments of the present disclosure.

FIG. 4 shows molecular orbitals of organic electroluminescent compounds represented by Formula (II).

### DETAILED DESCRIPTION OF THE DISCLOSURE

The following disclosure provides many different embodiments, or examples, for implementing different features of the provided subject matter. Specific examples of components and arrangements are described below to simplify the present disclosure. These are, of course, merely examples and are not intended to be limiting.

The present disclosure therefore provides an organic electroluminescent compound represented by the following Formula (I):

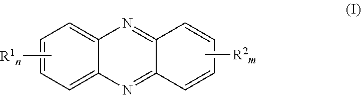

wherein each of R¹ and R² is independently an electron-donating group and each of n and m is independently an integer between 1 and 4.

In some embodiments, the compound is favorable to be used in an electroluminescent element of an organic electroluminescent device, which provides the organic electroluminescent device with advantages of a practical light emission efficiency and long service life. In some embodiments, the organic electroluminescent compounds of the present disclosure can be used in an electronic device such as an OLED, organic electroluminescent device or lighting device. In sonic embodiments, with the main body of the phenazine group being an electron-accepting group, and the R¹ and R² being electron-donating groups, the organic electroluminescent compound represented by the following Formula (I) has TADF properties. When the organic electroluminescent compound represented by Formula (I) is driven by a voltage, the compound can release energy in the form of fluorescence and delayed fluorescence.

The aforementioned “main body of the phenazine group” refers to the cyclic structure encircled by the carbon atoms and the nitrogen atom, but not including the hydrogen atoms or other substituents connecting with the carbon atoms.

In some embodiments, each of R¹ and R² is independently selected from the group consisting of the electron-donating groups represented by Formula (i), Formula (ii). Formula (iii) and Formula (iv):

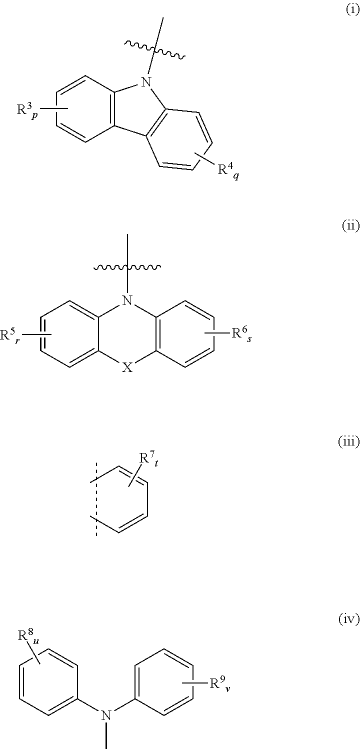

wherein each of R³, R⁴, R⁵, R⁶, R⁷, R⁸ and R⁹ is independently selected from the group consisting of hydrogen, an aryl group, a heteroaryl group, a C₁-C₆ alkyl group, a C₁-C₆ alkenyl group and a C₁-C₆ alkynyl group; each of p, q, r, s, t, u and v is independently an integer between 1 and 4; X is selected from the group consisting of NR¹⁰, O, S, CR¹¹₂, SiR¹²₂, PR¹³ and Se; and each of R¹⁰, R¹¹, R¹² and R¹³ is independently selected from the group consisting of hydrogen, an aryl group, a heteroaryl group, a C₁-C₆ alkyl group, a C₁-C₆ alkenyl group and a C₁-C₆ alkynyl group.

In some embodiments, the terms “alkyl”, “alkenyl” and “alkynyl” include both a linear structure and a branched structure.

In some embodiments, the term “aryl” refers to an organic radical derived from an aromatic hydrocarbon by removing one hydrogen atom. In some embodiments, the term “aryl” includes a monocyclic ring or fused ring each of whose rings has 4 to 7, preferably 5 or 6, ring backbone atoms, wherein the ring may be formed by linking two or more aryl groups to one another via one or more single bonds. In some embodiments, the term “aryl” includes phenyl, biphenyl, terphenyl, naphthyl, anthryl, indenyl, fluorenyl, phenanthrenvl, triphenylenyl, pyrenyl, perylenyl, chrysenyl, naphthacenyl, fluoranthenyl, etc. In some embodiments, the naphthyl includes 1-naphthyl and 2-naphthyl, the anthryl includes 1-anthryl, 2-anthryl and 9-anthryl, and the fluorenyl includes 1-fluorenyl, 2-fluorenyl, 3-fluorenyl, 4-fluorenyl and 9-fluorenyl.

In some embodiments, the term “heteroaryl” refers to an aryl having 1 to 4 heteroatoms selected from the group consisting of B, N, O, S, P(═O), Si and P, and carbon atoms as remaining ring backbone atoms other than the heteroatom. In some embodiments, the term “heteroaryl” refers to a monocyclic ring or fused ring condensed with at least one benzene ring. In sonic embodiments, the term “heteroaryl” may be formed by linking at least one heteroaryl group to another heteroaryl or aryl group via one or more single bonds. In some embodiments, the term “heteroaryl” may be a divalent aryl group whose ring backbone heteroatom is oxidized or quaternarized, for example, to form an N-oxide or a quaternary salt. In some embodiments, the term “heteroaryl” includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc. In some embodiments, the term “heteroaryl” includes a fused ring-type heteroaryl including benzofuranyl, benzothiophenyl, isobenzofuranyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl cinnolinyl, quinazolinyl, quinoxalinyl, carbazolvl, phenanthridinyl, benzodioxolyl, dibenzofuranyl, dibenzothiophenvl, etc., N-oxides thereof (for example, pyridyl N-oxide, quinolyl N-oxide), and quaternary salts thereof.

It should be particularly mentioned that each of the alkyl groups, the alkenyl groups, the alkynyl groups, the aryl groups and the heteroaryl groups may be a substituted or unsubstituted group.

in some embodiments, the aryl group is a substituted or non-substituted C₄-C₂₀ aryl group, and the heteroaryl group is a substituted or non-substituted C₄-C₂₀ heteroaryl group.

When an organic electroluminescent compound is excited. E_{S} represents the energy level of the singlet excited state, E_{T} represents the energy level of the triplet excited state, and ΔE_{ST} represents the difference between E_{S} and E_{T} (ΔE_{ST}=E_{S}−E_{T}), which is the energy gap between the singlet excited state and the triplet excited state. In some embodiments, E_{S} and E_{T} can be theoretically calculated by an electron density functional theory (B3LYP). In some embodiments, a difference between an energy level of a singlet excited state of the organic electroluminescent compound represented by Formula (I) and an energy level of a triplet excited state of the organic electroluminescent compound represented by Formula (I) is less than 0.20 eV. In brief, ΔE_{ST} of the organic electroluminescent compound represented by Formula (I) is less than 0.20 eV.

In some embodiments, the organic electroluminescent compound is represented by Formula (II):

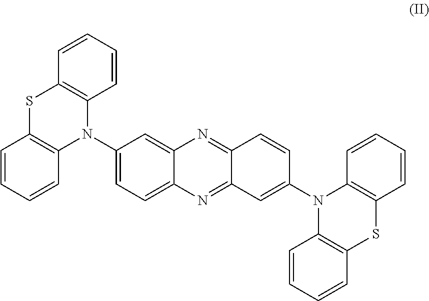 [SMILES: c1ccc2c(c1)Sc1ccccc1N2c1ccc2nc3cc(N4c5ccccc5Sc5ccccc54)ccc3nc2c1]

According to the result of theoretical calculation, the E_{S} of the organic compound represented by Formula (II) is 1.7792 eV, the E_{T} is 1.7134 eV, and the ΔE_{ST} is 0.0658, thus confirming that the compound is a TADF material. Further, the wavelength correspondent to the fluorescence peak (λₑₘ₁) is 696.86 nm, and the wavelength correspondent to the phosphorescence peak (λₑₘ₂) is 733.62 nm. [2017317292 0103] In some embodiments, the organic electroluminescent compound represented by Formula (II) emits red light under a bias voltage.

In the present disclosure, an OLED is disclosed. The OLED includes a pair of electrodes including an anode and a cathode, and an electroluminescent element disposed between the pair of electrodes, wherein the electroluminescent element comprises the aforementioned organic electroluminescent compound represented by Formula (I). The details of the compound have been recited previously and will not be repeated herein. In some embodiments, the organic electroluminescent compound represented by the formula (I) of the invention is useful as a light-emitting material of the OLED. Accordingly, the organic electroluminescent compound represented by the formula (I) of the invention may be effectively used as a light-emitting material in an electroluminescent element of an OLED. In some embodiments, the electroluminescent element comprises the aforementioned organic electroluminescent compound represented by Formula (II), The details of the compounds have been recited previously and will not be repeated herein.

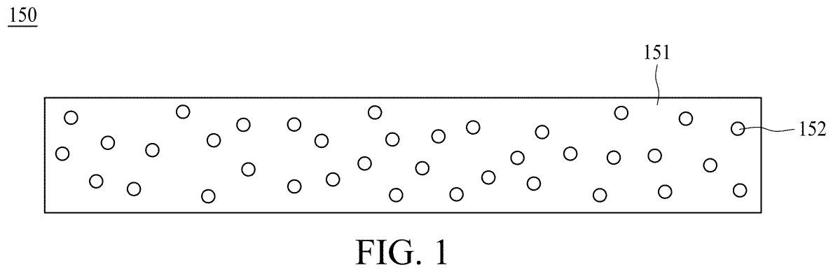

FIG. 1 is a schematic cross-sectional view illustrating an electroluminescent element of an OLED according to aspects of the present disclosure in some embodiments. In some embodiments, the electroluminescent element is an emitting layer 150. In some embodiments, the emission layer 150 emits red light under a bias voltage. In some embodiments, the OLED emits delayed fluorescent light. In some embodiments, the organic electroluminescent compound represented by the formula (I) includes a delayed fluorescent emitter emitting delayed fluorescent light. Thus, the present disclosure provides an invention relating to a delayed fluorescent emitter having the structure represented by the formula (I), and an invention relating to the use of the compound as the delayed fluorescent emitter. In some embodiments, the OLED that uses the compound as a light-emitting material has features that the device emits delayed fluorescent light and has a practical light emission efficiency and long service life.

In some embodiments, as shown in FIG. 1, the emitting layer 150 is made of host emitting material 151 doped with a suitable light-emitting material. In some embodiments, the emitting layer 150 includes a host emitting material 151 and a dopant 152.

In some embodiments, the dopant 152 is the aforementioned organic electroluminescent compound represented by the formula (I). In some embodiments, the dopant 152 is the aforementioned organic electroluminescent compound represented by the formula (II). Thus, the OLED including the light-emitting layer 150 features advantages of a practical light-emission efficiency and long service life. By adding the dopant 152 in the emitting layer 150, the energy of the host emitting material 151 can be transferred to the dopant 152, so that the light color and the luminous efficiency of the host emitting material 151 can be changed, thus broadening the application of the OLED.

In some embodiments, the host emitting material 151 is the aforementioned organic electroluminescent compound represented by the formula (I). In some embodiments, the host emitting material 151 is the aforementioned organic electroluminescent compound represented by the formula (II).

In the present disclosure, an organic electroluminescent device is disclosed. The organic electroluminescent device includes a cathode, an anode, and an organic layer disposed between the cathode and the anode and comprising an organic electroluminescent compound represented by Formula (I). The details of the compound have been recited previously and will not be repeated herein. In some embodiments, the organic layer disposed between the cathode and the anode comprises the aforementioned organic electroluminescent compound represented by the formula (II). In some embodiments, a plurality of organic layers are disposed between the cathode and the anode.

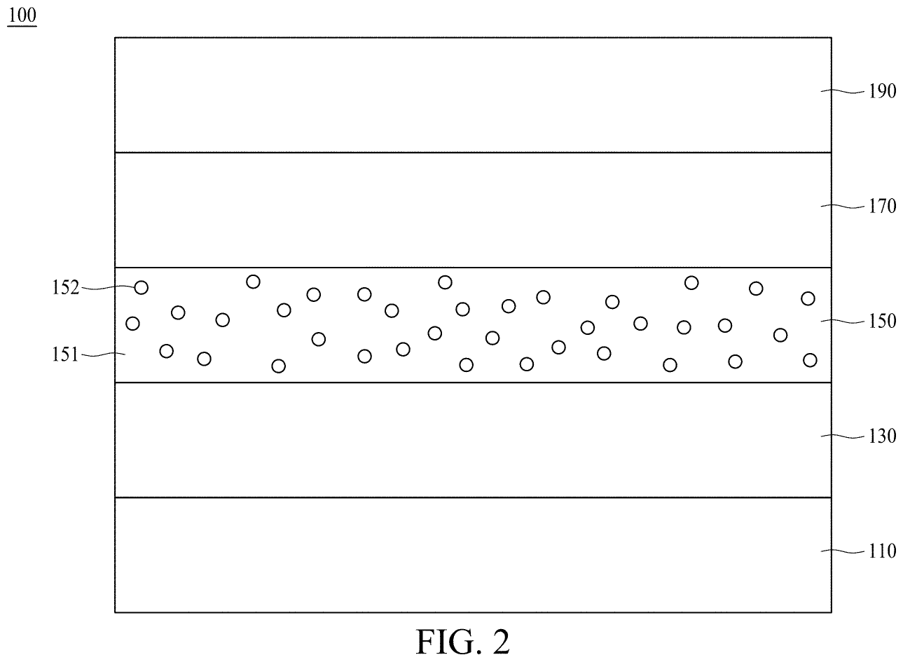

FIG. 2 is a schematic cross-sectional view illustrating an organic electroluminescent device according to aspects of the present disclosure in some embodiments. The configuration of the organic electroluminescent device of the present invention is not particularly limited. In some embodiments, as shown in FIG. 2, the organic electroluminescent device 100 includes an anode 110, a hole transport layer 130, an emitting layer 150, an electron transport layer 170 and a cathode 190. The details of the emitting layer 150 have been recited previously and will not be repeated herein.

In some embodiments, the anode 110 can be obtained from a conductor having high work function to facilitate the injection of holes in the emitting layer 150. The material of the anode 110 is, for instance, metal, metal oxide, a conducting polymer, or a combination thereof. In some embodiments, the metal is, for instance, Ni, Pt, V, Cr, Cu, Zn, Au, or an alloy thereof; the metal oxide is, for instance, zinc oxide, indium oxide, indium tin oxide (ITO), or indium zinc oxide (IZO); the combination of the metal and the oxide is, for instance, a combination of ZnO and Al or a combination of SnO₂ and Sb; and the conductive polymer is, for instance, poly(3-methylthiophene), poly(3,4-(ethylene-1,2-dioxy)thiophene (PEDT), polypyrrole, or polyaniline, but the invention is not limited thereto.

In some embodiments, the cathode 190 can be obtained from a conductor having low work function to facilitate the injection of electrons in the light-emitting layer 150. In some embodiments, the material of the cathode 190 is, for instance, metal or multilayer structure material. In some embodiments, the metal is, for instance, Mg, Ca, Na, Ti, In, Y, Li, Gd, Al, Ag, Sn, Pd, Cs, Ba, or an alloy thereof; the material of the multilayer structure is, for instance, LiF/Al, LiO₂/Al, LiF/Ca, LiF/Al, or BaF₂/Ca, but the invention is not limited thereto.

In some embodiments, the organic layers further include a hole-blocking layer (HBL) between the emitting layer 150 and the electron transport layer 170 or further include an electron-blocking layer (EBL) between the emitting layer 150 and the hole transport layer 130. In some embodiments, due to the longer service life and the diffusion length of triplet excitons compared to those of singlet excitons, when the organic electroluminescent device is a phosphorescent organic electroluminescent device, the additional hole-blocking layer and electron-blocking layer are especially needed. The purpose of the use of hole-blocking layer or electron-blocking layer is to confine the recombination of injected holes and electrons and the relaxation of created excitons within the emitting layer, thus improving the device's efficiency. To serve such functions, the hole-blocking materials or electron-blocking materials must have HOMO and LUMO energy levels suitable to block holes or electrons from being transported from the emitting layer 150 to the electron transport layer 170 or the hole transport layer 130.

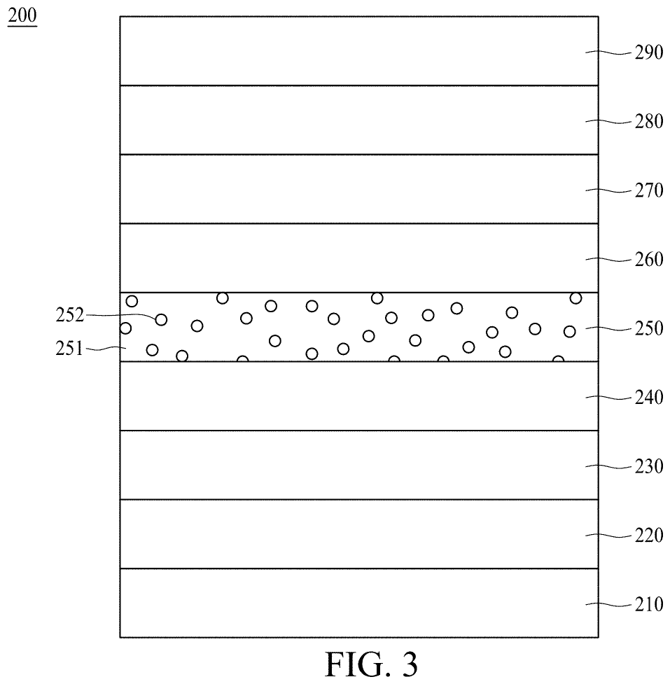

FIG. 3 is a schematic cross-sectional view illustrating an organic electroluminescent device according to aspects of the present disclosure in some embodiments. In sonic embodiments, as shown in FIG. 3, the organic electroluminescent device 200 includes an anode 210, a hole-injecting layer (HIL) 220, a hole transport layer 230, an electron-blocking layer 240, an emitting layer 250, a hole-blocking layer 260, an electron transport layer 270, an electron-injecting layer (EIL) 280, and a cathode 290. In some embodiments, the hole-injecting layer 220, the hole transport layer 230, the electron-blocking layer 240, the emitting layer 250, the hole-blocking layer 260, the electron transport layer 270, the electron-injecting layer 280, and the cathode 290 are sequentially configured on top of the anode 210.

In some embodiments, the organic electroluminescent compound represented by Formula (I) is a host emitting material 251 of the emitting layer 250 of the organic electroluminescent device 200. In some embodiments, the organic electroluminescent compound represented by Formula (II) is the host emitting material 251 of the emitting layer 250 of the OLED device 200.

In some embodiments, the organic compound represented by Formula (I) is a dopant 252 of the emitting layer 250 of the OLED device 200. In some embodiments, the organic electroluminescent compound represented by Formula (II) is the dopant 252 of the emitting layer 250 of the OLED device 200.

In some embodiments, the organic electroluminescent compound represented by Formula (I) is a hole transport material of the hole transport layer 230 of the organic electroluminescent device 200. In some embodiments, the organic electroluminescent compound represented by Formula (II) is the hole transport material of the hole transport layer 230 of the organic electroluminescent device 200.

In some embodiments, the organic electroluminescent compound represented by Formula (I) is an electron transport material of the electron transport layer 270 of the organic electroluminescent device 200. In some embodiments, the organic electroluminescent compound represented by Formula (II) is the electron transport material of the electron transport layer 270 of the organic electroluminescent device 200.

In some embodiments, the organic electroluminescent compound represented by Formula (I) is a hole-blocking material of the hole-blocking layer 260 of the organic electroluminescent device 200. In some embodiments, the organic electroluminescent compound represented by Formula (II) is the hole-blocking material of the hole-blocking layer 260 of the organic electroluminescent device 200.

In some embodiments, the organic electroluminescent compound represented by Formula (I) is an electron-blocking material of the electron-blocking layer 240 of the organic electroluminescent device 200. In some embodiments, the organic electroluminescent compound represented by Formula (II) is the electron-blocking material of the electron-blocking layer 240 of the organic electroluminescent device 200.

In some embodiments, the organic electroluminescent compound represented by Formula (I) is a hole-injecting material of the hole-injecting layer 220 of the organic electroluminescent device 200. In some embodiments, the organic electroluminescent compound represented by Formula (II) is the hole-injecting material of the hole-injecting layer 220 of the organic electroluminescent device 200.

In some embodiments, the organic electroluminescent compound represented by Formula (I) is an electron-injecting material of the electron-injecting layer 280 of the organic electroluminescent device 200. In some embodiments, the organic electroluminescent compound represented by Formula (II) is the electron-injecting material of the electron-injecting layer 280 of the organic electroluminescent device 200.

In the present disclosure, a method of preparing the organic electroluminescent compound represented by Formula (I) is disclosed. In some embodiments, the organic electroluminescent compound represented by Formula (I) is prepared by the following method.

First, the main body of the phenazine group of the organic electroluminescent compound represented by Formula (I) can be synthesized according to Scheme 1:

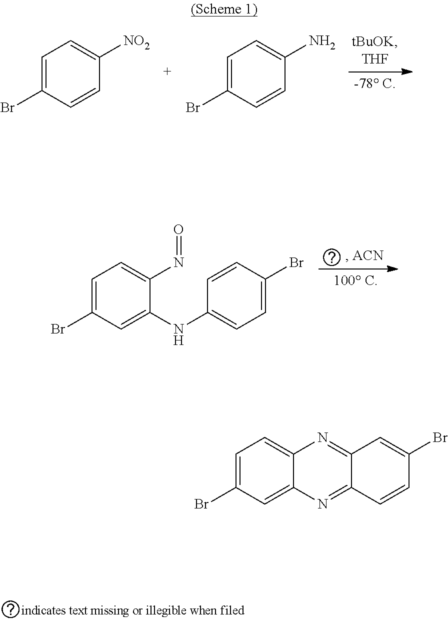 [SMILES: O=[N+]([O-])c1ccc(Br)cc1 ; Nc1ccc(Br)cc1 ; O=Nc1ccc(Br)cc1Nc1ccc(Br)cc1 ; Brc1ccc2nc3cc(Br)ccc3nc2c1]

In Scheme 1, the main body of the phenazine group is synthesized by two steps, the first step and the second step.

In the first step, N-4-bromophenyl-2-nitroso-4-bromoaniline is synthesized. as follows. Aniline (6 mmol) in THF (5 mL) and nitroarene (6 mmol) in THF (5 mL) were sequentially and dropwisely added into a cooled solution of t-BuOk (18 mmol, 2.02 g) in THF (25 mL) at −78° C. to form a reaction mixture. The reaction mixture was stirred at −78° C. for 1 hour. The reaction mixture was then poured into a concentrated NH₄Cl solution (ca. 70 mL) and extracted with EtOAc for a few times. The organic layer of the mixture was washed with water and brine, and dried over Na₂SO₄ to provide an extract. The solvent of the extract was evaporated, and the remaining residue was purified by silica gel chromatography (1:9 EA/Hex) to obtain the N-4-bromo-phenyl-2-nitroso-4-bromoaniline,

In the second step, N-aryl-2-nitrosoanilines is cyclized as follows. N, O bis(trimethylsilypacetamide (BSA) (1.2 mL, 5 mmol) was added to a solution of nitrosoaniline (1 mmol) in MeCN (4 mL) to form a reaction mixture. The reaction mixture was added into a sealed tube. The sealed tube was then heated to 100° C. for 12 hours. The reaction mixture was filtered to obtain a precipitate, and the precipitate was washed with cold MeOH, then dried under vacuum to obtain the main body of the phenazine group of the organic electroluminescent compound represented by Formula (I).

Second, the main body of the phenazine group is coupled with electron donors. In some embodiments, the organic electroluminescent compound represented by Formula (II) can be synthesized according to Scheme 2:

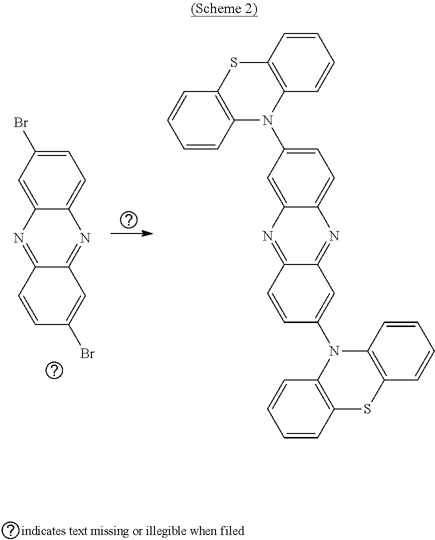 [SMILES: Brc1ccc2nc3cc(Br)ccc3nc2c1 ; c1ccc2c(c1)Sc1ccccc1N2c1ccc2nc3cc(N4c5ccccc5Sc5ccccc54)ccc3nc2c1]

Dibromophenazine (1 mmol), nitrogen donor (2 mmol), copper powder (4 mmol) and K₂CO₃ (5 mmol) were added in a flask. Dichlorobenzene (5 mL) was further added into the flask under nitrogen atmosphere and the flask was heated to 190° C. in an oil bath for 24 hours to obtain a reaction mixture. The reaction mixture was filtered to obtain a solution. The organic solvent of the solution was removed under vacuum to obtain a residue. The residue was purified by silica gel chromatography to obtain the organic electroluminescent compound represented by Formula (II).

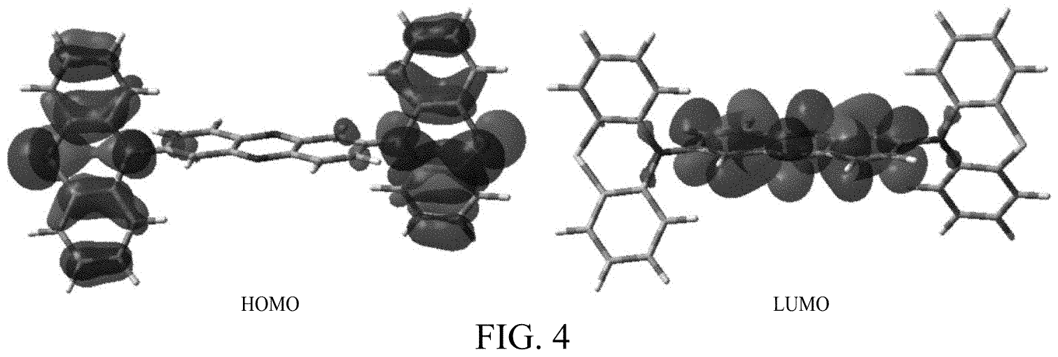

FIG. 4 shows molecular orbitals of the organic electroluminescent compound represented by Formula (II). FIG. 4 is obtained from the calculation results of time-dependent density functional theory, and shows the distribution of HOMO and LUMO. As shown in FIG. 4, the HOMO are mainly distributed over the electron-donating groups, the LUMO are mainly distributed over the electron-accepting group composed of the main body of the phenazine group, and there is a small overlap between the HOMO and the LUMO, which shows that the organic compound represented by Formula (II) has TADF property.

In view of the aforesaid, the organic electroluminescent compound represented by Formula (I), which contains a main body of the phenazine group, is useful as a light-emitting material. Accordingly, the organic electroluminescent compound represented by Formula (I) of the present invention may be effectively used as a light-emitting material of an organic light-emitting device, such as an organic electroluminescent device. The organic electroluminescent compound of the present invention emits delayed fluorescent light, and thus may be capable of providing an organic light-emitting device having a practical efficiency and long service life.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An organic electroluminescent compound represented by Formula (I): 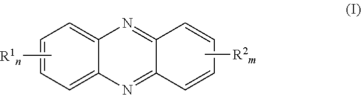
wherein each of R¹ and R² is independently an electron-donating group and each of n and m is independently an integer between 1 and 4.

2. The organic electroluminescent compound of claim 1, wherein each of R¹ and R² is independently selected from the group consisting of the electron-donating groups represented by Formula (i), Formula (ii), Formula (iii) and Formula (iv): 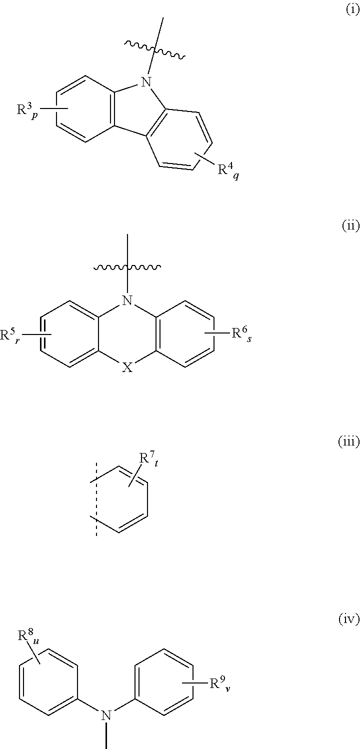
wherein each of R³, R⁴, R⁵, R⁶, R⁷, R⁸ and R⁹ is independently selected from the group consisting of hydrogen, an aryl group, a heteroaryl group, a C₁-C₆ alkyl group, a C₁-C₆ alkenyl group and a C₁-C₆ alkynyl group; each of p, q, r, s, t, u and v is independently an integer between 1 and 4; X is selected from the group consisting of NR¹⁰, O, S, CR¹¹₂, SiR¹²₂, PR¹³ and Se; and each of R¹⁰, R¹¹, R¹² and R¹³ is independently selected from the group consisting of hydrogen, an aryl group, a heteroaryl group, a C₁-C₆ alkyl group, a C₁-C₆ alkenyl group and a C₁-C₆ alkynyl group.

3. The organic electroluminescent compound of claim 2, wherein the aryl group is a substituted or non-substituted C₄-C₂₄ aryl group, and the heteroaryl group is a substituted or non-substituted C₄-C₂₄ heteroaryl group.

4. The organic electroluminescent compound as claimed in claim 1, wherein the organic compound is represented by Formula (II):

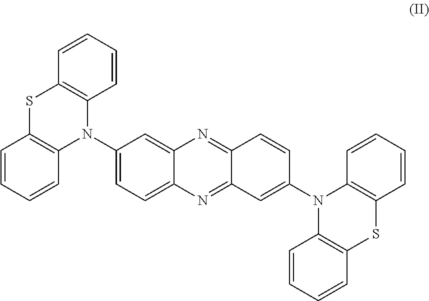 [SMILES: c1ccc2c(c1)Sc1ccccc1N2c1ccc2nc3cc(N4c5ccccc5Sc5ccccc54)ccc3nc2c1]

5. The organic electroluminescent compound as claimed in claim 1, wherein a difference between an energy level of a singlet excited state of the organic electroluminescent compound represented by Formula (I) and an energy level of a triplet excited state of the organic electroluminescent compound represented by Formula (I) is less than 0.20 eV.

6. An organic light-emitting device, comprising:
a pair of electrodes; and
an electroluminescent element disposed between the pair of electrodes, wherein the electroluminescent element comprises an organic electroluminescent compound represented by Formula (I): 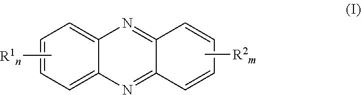
wherein each of R¹ and R² is independently an electron-donating group and each of n and m is independently an integer between 1 and 4.

7. The organic light-emitting device according to claim 6, wherein the organic light-emitting device emits delayed fluorescent light.

8. An organic electroluminescent device, comprising:
a cathode;
an anode; and
an organic layer disposed between the cathode and the anode and comprising an organic electroluminescent compound represented by Formula (I): 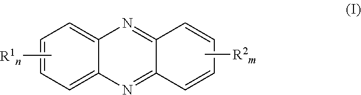
wherein each of R¹ and R² is independently selected from the group consisting of the electron-donating groups represented by Formula (i), Formula (ii), Formula (iii) and Formula (iv): 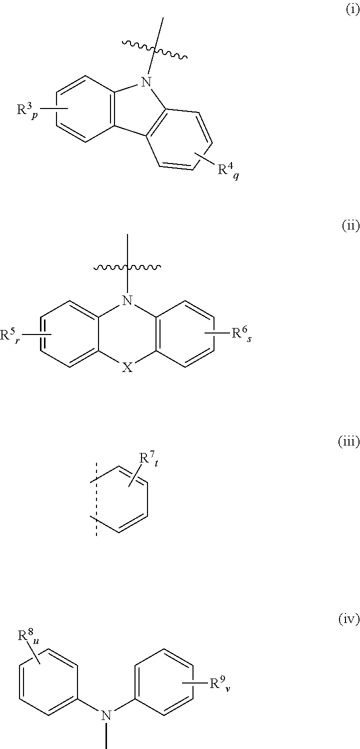
wherein each of R³, R⁴, R⁵, R⁶, R⁷, R⁸ and R⁹ is independently selected from the group consisting of hydrogen, an aryl group, a heteroaryl group, a C₁-C₆ alkyl group, a C₁-C₆ alkenyl group and a C₁-C₆ alkynyl group; each of p, q, r, s, t, u and v is independently an integer between 1 and 4; X is selected from the group consisting of NR¹⁰, O, S, CR¹¹₂, SiR¹²₂, PR¹³ and Se; and each of R¹⁰, R¹¹, R¹² and R¹³ is independently selected from the group consisting of hydrogen, an aryl group, a heteroaryl group, a C₁-C₆ alkyl group, a C₁-C₆ alkenyl group and a C₁-C₆ alkynyl group; and
each of n and m is independently an integer between 1 and 4.

9. The organic electroluminescent device of claim 8, wherein the organic layer comprises an emitting layer, and the organic electroluminescent compound represented by Formula (I) is a host emitting material.

10. The organic electroluminescent device of claim 8, wherein the organic layer comprises an emitting layer, and the organic electroluminescent compound represented by Formula (I) is a dopant.

11. The organic electroluminescent device of claim 8, wherein the emission layer emits red light under a bias voltage.

12. The organic electroluminescent device of claim 8, further comprising an emitting layer disposed between the cathode and the anode.

13. The organic electroluminescent device of claim 12, wherein the organic layer comprises a hole transport layer disposed between the emitting layer and the anode, and the organic electroluminescent compound represented by Formula (I) is a hole transport material.

14. The organic electroluminescent device of claim 12, wherein the organic layer comprises an electron transport layer disposed between the emitting layer and the cathode, and the organic electroluminescent compound represented by Formula (I) is an electron transport material.

15. The organic electroluminescent device of claim 12, wherein the organic layer comprises a hole-blocking layer disposed between the emitting layer and the cathode, and the organic electroluminescent compound represented by Formula (I) is a hole-blocking material.

16. The organic electroluminescent device of claim 12, wherein the organic layer comprises an electron-blocking layer disposed between the emitting layer and the anode, and the organic electroluminescent compound represented by Formula (I) is an electron-blocking material.

17. The organic electroluminescent device of claim 12, wherein the organic layer comprises a hole-injecting layer disposed between the emitting layer and the anode, and the organic electroluminescent compound represented by Formula (I) is a hole-injecting material.

18. The organic electroluminescent device of claim 12, wherein the organic layer comprises an electron-injecting layer disposed between the emitting layer and the cathode, and the organic electroluminescent compound represented by Formula (I) is an electron-injecting material.
